(19) United States
(12) Patent Application Publication
(10) Pub. No.: US 2005/0053580 A1
(43) Pub. Date: Mar. 10, 2005

(54) Use of microbiology non-viral substances for treating acne

(76) Inventor: Christos C. Zouboulis, Berlin (DE)

Correspondence Address: MAYER, BROWN, ROWE & MAW LLP, 190 SOUTH LASALLE ST, CHICAGO, IL, 60603-3441, US

(21) Appl. No.: 10/487,618

(22) PCT Filed: Aug. 23, 2002
(86) PCT No.: PCT/EP02/09452

(30) Foreign Application Priority Data
Aug. 23, 2001 (DE) 101 41 443.9

(51) Int. Cl. A61K 48/00

(52) U.S. Cl. 424/93.2; 514/44

(57) ABSTRACT

This is to describe the use of at least one molecular-biologically prepared non-viral active agent (antisense oligonucleotides, ribozymes, stabilized ribozymes, aptamers, mirror aptamers, chimeric RNA/DNA oligonucleotides, naked plasmid-DNA or liposomally encapsulated DNA), especially an antisense oligonucleotide having specificity for a nucleic acid target sequence of the genes, which codes for steroid hormone receptors and/or steroid hormone metabolizing enzymes, especially for the androgen receptor and/or the 5α-reductase, for the treatment of acne and acneiform dermatoses including rosacea. For this purpose, preferably specific antisense oligonucleotides and degenerated sequences thereof are selected and preferably introduced by a liposome-mediated transfection into the target cells, especially human sebocytes and/or keratinocytes. In these target cells, a specific inhibition of the androgen-induced, especially of the testosterone-induced stimulation of the sebocyte and/or keratinocyte proliferation by the described antisense oligonucleotides has been determined and evaluated. The effects according to the invention have been detected by way of example with the aid of the sebocyte cell line SZ95 and primary keratinocytes of the skin.

The present invention relates to the use of molecular-biologically prepared non-viral active agents (antisense oligonucleotides, ribozymes, stabilized ribozymes, aptamers, mirror aptamers, chimeric RNA/DNA oligonucleotides, naked plasmid DNA, liposomally encapsulated DNA) for the treatment of acne and acneiform dermatoses including rosacea. The active agents can be administered in a pharmaceutical composition according to any conventional application route.

It is assumed that acne lesions occur due to the interaction of interrelated complex processes: an increased production and excretion of the sebum (Cunliffe W J, Shuster S: Pathogenesis of acne. Lancet. 1969; 1(7597): 685-687), a ductal cornification (Holmes R L, Williams M, Cunliffe W J: Pilosebaceous duct obstruction and acne. Br J Dermatol. 1972; 87: 327-332 and 35), an abnormal number and function of Propionibacterium acnes (Cunliffe W J, Clayden A D, Gould D, Simpson N B: Acne vulgaris-its aetiology and treatment. A review. Clin Exp Dermatol. 1981; 6: 461-469) and the production of inflammatory mediators which result in the formation of papules, pustules and temporarily deep inflammatory lesions.

The increased production and excretion of sebum in the case of acne is caused, inter alia, by an acceleration of the differentiating cycle of the sebaceous gland cells (sebocytes). The androgens are the most important regulators of the sebocytes, both in the ontogenetic development and in the control of the activity of the differentiated sebocytes, they increase lipogenesis, proliferation and terminal differentiation of sebocytes as well as some keratinocyte populations in the skin. The regulation is effected by the bonding of an androgen-androgen receptor complex to competent positions of the genomic DNA, whereby the synthesis of specific proteins is initiated. In this context, testosterone and 5α-dihydrotestosterone (DHT) are the two most important representatives of the androgens; wherein DHT bonds with a definitely higher affinity to the androgen receptor than testosterone. DHT is synthesized in an intracellular way from the testosterone with the aid of the 5α-reductase enzyme. This metabolic function takes place in the skin especially in the sebocytes where recently a superproportional 5α-reductase activity was localized. Two genes were found which code for two different isoenzymes (type-1 and type-2) of the 5α-reductase enzyme (gene base locus: HUM5AR and HUMSRDA), the isoenzyme type-1 being especially active in the sebaceous glands.

Moreover the largest number of intracellular androgen receptors of the skin was established in the sebocytes. For these reasons, great importance is attributed to the inhibition of the 5α-reductase activity (especially of the type-1 isoenzyme) and of the androgen receptor in the skin for the inhibition of the terminal differentiation of the sebocytes and thus for the treatment of seborrhea and acne. First experimental and clinical results when applying specific inhibitors of the 5α-reductase isoenzyme type-1 and of the androgen receptor confirm this assumption, however for this purpose the systemic administration of the respective active agents which is undesired due to its strong effects on the entire body is necessary.

For a short time tests have been made to carry out a well-directed therapy of different diseases by means of molecular-biologically prepared agents. For instance, novel drugs can be developed by the antisense technology. Traditional xenobiotics act on proteins which have resulted in a disease in the organism. Antisense molecules work on a genetic level by specifically blocking the translation of the protein which causes a disorder. Therefore, drugs on the basis of antisense have the potential to act in a more selective way and, thus, are more effective and less toxic. During the past few years, the development of effector molecules on the basis of nucleic acid, called aptamers, has been successively employed for inhibiting and/or activating different enzymes and receptors [Ruckman J. et al.: 2′-fluoropyrimidine RNA-based aptamers to the 165-amino acid form or vascular endothelial growth factor (VEGF 165). Inhibition of receptor binding and VEGF-induced vascular permeability through interactions requiring the exon 7-encoded domain. J. Biol. Chem. 1998; 273: 20556-20567].

At present 15 clinical studies are being carried out in which antisense oligonucleotides are employed. The systemic treatment with antisense oligonucleotides is still difficult due to the possible cytotoxicity of the antisense oligonucleotides and the carrier molecules such as cationic lipids. The treatment of pathologic conditions impairing the skin can be partly improved by local application. However, the success of the antisense strategy has been restricted so far by the low receptivity of the transfection reagent and the insufficient intracellular compartmenting. Human epithelium cells, in particular those of the skin, are the preferred target tissue for judging the receptivity and the effect of antisense oligonucleotides for therapeutic and diagnostic purposes in vitro and in vivo.

The biological effect of the antisense oligonucleotides is influenced by various factors such as the local concentration of the oligonucleotide, the initial concentration of the oligonucleotide, the local concentration of the oligonucleotide at the target tissue, the penetration of the oligonucleotide through the cell membrane, the lipid/oligonucleotide ratio and the electric charge resulting therefrom, the rate of decomposition of the oligonucleotide and the DNA and/or RNA prolongation rate of the target gene. Molecular-biologically prepared drugs on the basis of antisense, ribozyme, stabilized ribozyme, aptamer, mirror aptamer, chimeric RNA/DNA oligonucleotide, naked plasmid-DNA and liposomally encapsulated DNA thus have the potential to act more selectively and thus more effectively and in a less toxic way than conventional therapeutics.

The development of antisense oligonucleotides with specificity for target sequences of genes which code for the androgen receptor and 5α-reductase is known from the U.S. Pat. No. 5,877,160. The progression of the male alopecia could be inhibited by a reduced production of the transcription products. By administering specifically synthesized antisense oligonucleotides (oligoribonucleotides and phosphorothioate-oligonucleotides) against the androgen receptor and 5α-reductase the concentration of 5α-dihydrotestosterone (DHT) bonded to the androgen receptor in hair follicles could be definitely reduced without influencing the testosterone synthesis and/or the metabolism thereof in other tissues. The androgen binding capacity for DHT in skin cells was substantially reduced after application of oligonucleotide.

Thus it is the object of the invention to enlarge the possible therapies against acne and to possibly improve the effect while simultaneously reducing side effects without accepting the drawbacks due to a systemic administration.

The object is achieved, according to the invention, by using one or plural molecular-biologically prepared non-viral active agents, especially antisense oligonucleotides with specificity for target sequences in genes coding for the androgen receptor and/or the 5α-reductase for the treatment of acne. Apart from the especially preferred antisense oligonucleotides, also ribozymes, stabilized ribozymes, aptamers, mirror aptamers, chimeric RNA/DNA oligonucleotides, naked plasmid-DNA or liposomally encapsulated DNA can be used.

According to the concept of action of a particularly preferred embodiment of the invention, antisense oligonucleotides were selected and designed which are complementary to an RNA sequence that is transcribed from a target gene into a single strand DNA target sequence or a single strand RNA or DNA within a duplex molecule. Such antisense oligonucleotides preferably comprise at least one of the following sequences:

5′-CTG CAC TTC CAT CCT TGA GCT TGG C-3′

a phosphorothioate-antisense-oligonucleotide (anti-DNA) and

5′-GCC AAG CUC AAG GAU GGA AGU GCA G-3′

a methylribosyl-antisense-oligonucleotide (anti-RNA),
corresponding to the nucleotide positions of the androgen receptor gene 13 to 12 (initial transcription position).

The thus designed duplex molecules inhibit the translation, the processing or the transport of mRNA sequence or result in digestion by the ribonuclease-H enzyme. Ribonuclease-H degrades the RNA strand of an RNA-DNA duplex molecule.

The oligonucleotides interact with other biomolecules such as lipids, carbohydrates and proteins. They induce non-antisense effects which incorrectly can be interpreted as antisense effects. In order to safeguard the specificity of our antisense oligonucleotides against the androgen receptor, we have also designed mismatch oligonucleotides as follows:

5′-CTG CCC TTC AAT CCC TGA GTT TGG C-3′

Likewise degenerated sequences or similar sequences having a comparable specificity can be used for the intended target sequences.

Further preferably the antisense oligonucleotides employed according to the invention have specificity for epithelial cells, especially human epithelial cells. The use according to the invention is especially suited in sebocytes and epidermal keratinocytes. In a further subject matter of the invention one or more of the antisense oligonucleotides, ribozymes, stabilized ribozymes, aptamers, mirror aptamers, chimeric RNA/DNA oligonucleotides, naked plasmid DNA or liposomally encapsulated DNA are used in a suited pharmaceutical composition with usual pharmaceutically acceptable carriers and optionally further adjuvants. The administration in the form of a lipid-mediated transfection of sebocytes and/or keratinocytes is especially suited. What is especially preferred in this respect is the uptake of antisense oligonucleotides by means of a liposome-mediated particularly cationic liposome-mediated transfection of the target tissue.

It was found within the scope of the invention that by the active agents used according to the invention, especially the antisense oligonucleotides, the androgenic stimulation of the sebaceous gland activity can be effectively inhibited and therefore molecular-biologically prepared molecules (antisense oligonucleotides, ribozymes, stabilized ribozymes, aptamers, mirror aptamers, chimeric RNA/DNA oligonucleotides, naked plasmid DNA or liposomally encapsulated DNA) can be used as excellent active agents for the treatment of acne.

Testosterone is the substantial circulating androgen in the body, but it has a low activity in skin cells. A much more active metabolite is 5α-dihydrotestosterone (DHT). DHT is formed after reduction of testosterone by the 5α-reductase enzyme. The effects of androgens are mediated by bonding the ligand (primarily DHT) to the androgen receptor which is either present in the nucleus or is transferred to the cytoplasm. The androgen receptor belongs to the sub-family of nuclear receptors which includes adrenocortical steroid, thyroid hormone, retinoic acid, vitamin D, estrogen, progesterone and PPA receptors whose activity is controlled by the close and specific bond of the ligand.

It was found, according to the present invention, that the biological activity of the androgen receptor and the 5α-reductase in native and in androgen receptor antisense transfected sebocytes and/or keratinocytes can be inhibited after their stimulation with androgens. The transient reduction of the androgen receptor expression as well as the expression of 5α-reductase after transient transfection of skin cells such as sebocytes and keratinocytes demonstrates that the use of the antisense oligonucleotides according to the invention is a highly efficient means for a therapeutic treatment of acne and of acneiform dermatoses including rosacea.

It is assumed that the surprising effectiveness of the antisense oligonucleotides employed according to the invention vis-a-vis the androgen receptor and the 5α-reductase has to be attributed to the specific bonding to a RNA sequence transcribed by the target gene and the formation of a duplex strand. This duplex strand inhibits the translation, the processing and the transport of the mRNA sequence or results in a digestion by ribonuclease-H. The oligonucleotides used according to the invention may activate the ribonuclease-H. The specific bonding of the antisense oligonucleotides used according to the invention can also be effected at a double-strand nucleic acid while forming a triplex complex.

Hereinafter the present invention will be described in detail with reference to preferred embodiments in connection with the accompanying figures in which:

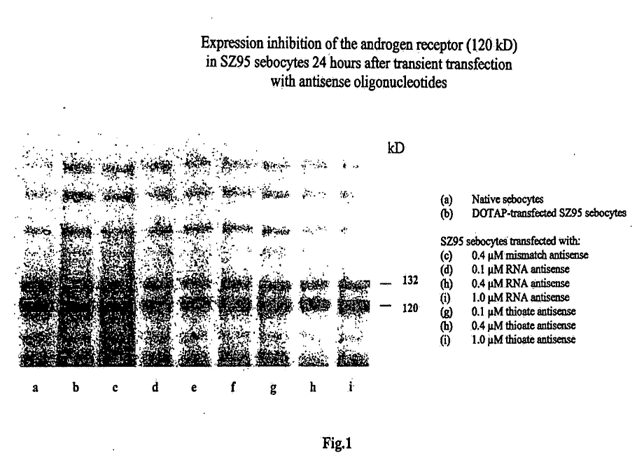

FIG. 1 shows an illustration of the expression inhibition of the androgen receptor (120 kD) in SZ95 sebocytes 24 hours after a transient transfection with antisense oligonucleotides. The figure shows Western blots in which the respective separated protein was incubated with polyclonal anti-androgen receptor antibody from hare and with immunoglobulin G directed against hare which was conjugated with secondary horse radish peroxidase. The blots were evaluated by chemiluminescence and the intensities of the androgen receptor protein bands were determined densitometrically by the computer program TINA 2.0.

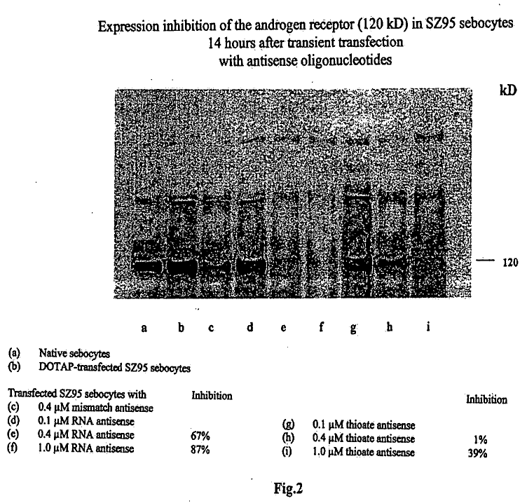

FIG. 2 shows an illustration of the expression inhibition of the androgen receptor (120 kD) in SZ95 sebocytes 14 hours after a transient transfection with antisense oligonucleotides by the afore-described detection system, and

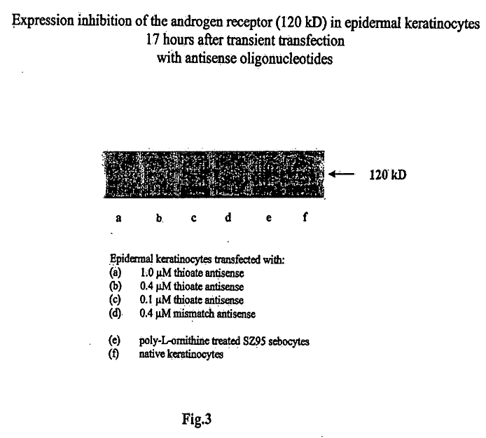

FIG. 3 is an explanation of the expression inhibition of the androgen receptor (120 kD) in epidermal keratinocytes 17 hours after a transient transfection with antisense oligonucleotides by the afore-described detection system,

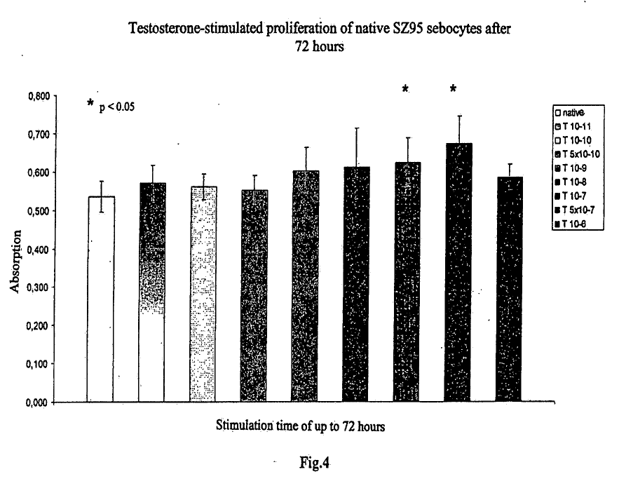

FIG. 4 shows an overview of the testosterone-stimulated proliferation of native SZ95 sebocytes after a stimulation period of 72 hours at different concentrations of testosterone,

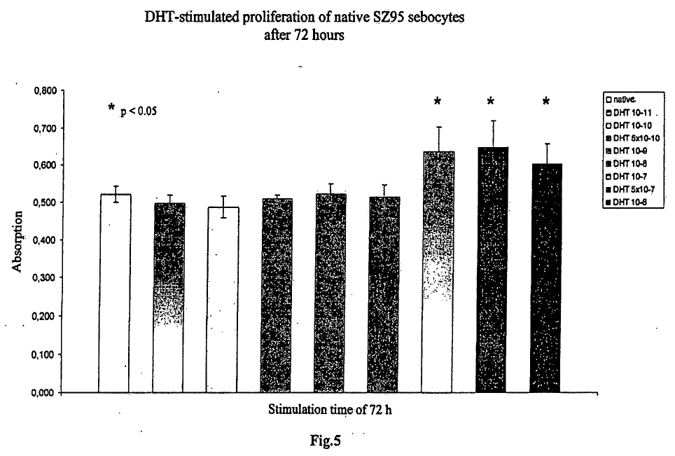

FIG. 5 shows an overview of the DHT-stimulated proliferation of native SZ95 sebocytes after a stimulation period of 72 hours at different concentrations of DHT,

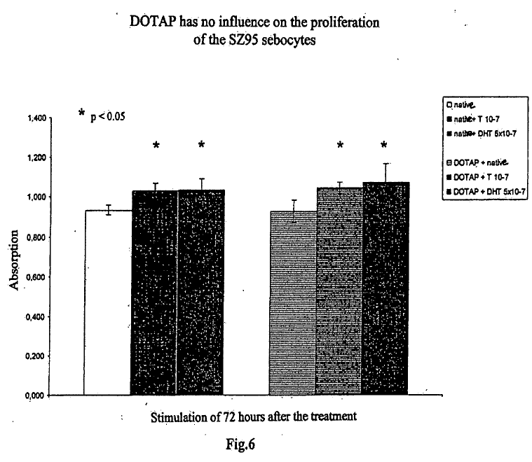

FIG. 6 is an overview of the proliferation rates of SZ95 sebocytes treated with DOTAP after a stimulation period of 72 hours,

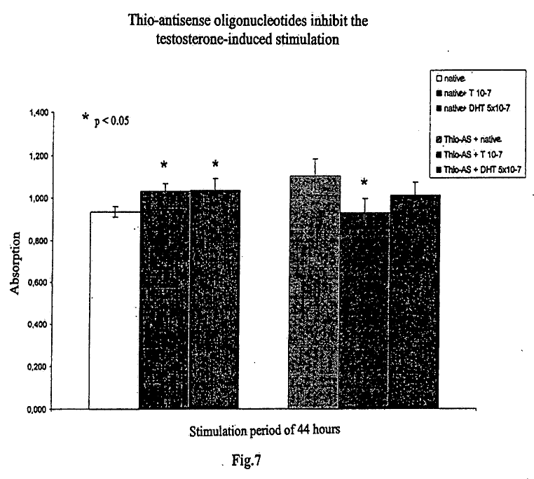

FIG. 7 is an overview of the inhibition of the testosterone-induced stimulation of the SZ95 sebocytes proliferation by thio-antisense oligonucleotides in different concentrations of testosterone and/or DHT after a stimulation period of 44 hours, and

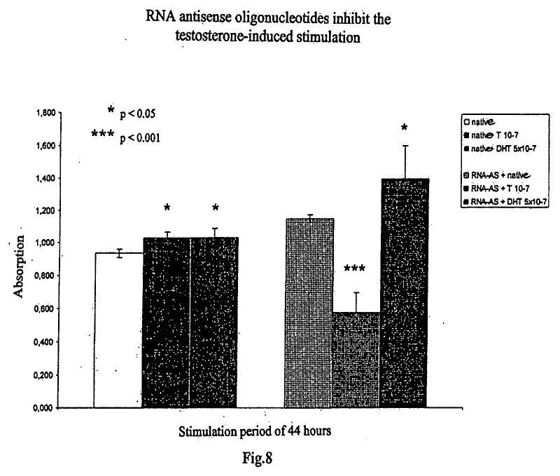

FIG. 8 is an overview of the inhibition of the testosterone-induced stimulation of the SZ95 sebocytes proliferation by RNA-antisense oligonucleotides in different concentrations of testosterone and/or DHT after a stimulation period of 44 hours.

The oligonucleotides can have a charged or an uncharged strand. Neutral oligomers are preferred, because they are easily absorbed by the skin when they are topically applied. They are quickly released from the plasma and discharged into the urine. The oligonucleotides used according to the invention furthermore can be preferably chemically modified. There are especially preferred neutral methyl phosphonate oligomers which merely have methyl phosphonate internucleosidyl bonds. The presence of methyl phosphonate or other neutral internucleotide bonds in the oligomer provide an exonuclear resistance. The use of nucleotide units having 2′-O-alkyl- or 2′-halo- and especially 2′-O-fluoro- or 2′-methyl-ribosyl units in the neutral oligomers can advantageously improve the hybridization of the oligomer with the complementary target sequence thereof. Further preferred oligomers include phosphorate thioate oligonucleotides. Oligonucleotides of this kind have a longer half-life in vivo, because their neutral structure reduces the rate of the nuclease degradation, while the cleaving or cross-linking unit can promote the inactivation of the polynucleotide target sequences.

A particularly efficient measure of introducing the oligonucleotides according to the invention into the epithelial target cells is the liposome transfection. The reception of the antisense molecules in the cells is increased by this measure. The mixture of liposome transfection reagents with the oligonucleotides results in stable complexes which can be directly added to the cell culture medium. These complexes bond to the cell surface, merge with the cell membrane and discharge the negatively charged oligonucleotides into the cytoplasm. What is especially efficient is the transfection while using cationic lipids which can bring about an increased transfection rate of the antisense oligonucleotides. When selecting the transfection reagent, it is especially important to bring about a non-toxic uptake of the oligonucleotides in the cultivated epithelial cells, especially sebocytes and keratinocytes.

In accordance with a particularly preferred embodiment of the invention, polycationic lipids, especially the polycation DOTAP, are used for the transfection of SZ95 sebocytes and especially the polycation L-ornithine is used for the transfection of keratinocytes. Using these lipids there are provided effective transfection systems without a substantial cytotoxicity. The reduced cytotoxicity can be controlled by varying the conditions of culture such as the confluence, the oligonucleotide concentration and the charge ratio between the lipid and the oligonucleotide. An adjustment of the lipid to oligonucleotide ratio is desired. A sufficient lipid quantity is required for preparing complexes with the oligonucleotide so as to provide a positive total charge. It is assumed that the positive charge of the lipid oligonucleotide complex facilitates the interaction with the negatively charged cell surface. A surplus of lipid in turn increases the toxic effect on the cells, however, and thus entails a modification of the cell morphology, a reduced growth and finally cytolysis. For SZ95 sebocytes a charge ratio of 2:1 of DOTAP and for keratinocytes a charge ratio of approx. 1:1 of L-ornithine has proved to be especially suited.

The active agent can be administered by usual ways of application, e.g. in a systemic way, by the intestinal route, especially orally or rectally, by the transdermal route, nasally by means of inhalation as well as by the parenteral route, especially by means of injection (subcutaneously, by the intramuscular or intravenous route) or the like. There can be treated human beings and animals such as mammals and rodents. In order to keep the systemic disturbance by the use of the active agent as low as possible or exclude it, respectively, the local and especially the topical application is preferred. For this purpose carrier-free systems (in ointments, creams, gels, oils, emulsions, lotions, pastes or solutions and/or other compositions) or systems based on carriers such as pavements, dressing material etc. can be applied. A particularly preferred possible use consists in the application of the oligonucleotide preparation in liposomes or in liposomes which are absorbed in a different composition.

The antisense oligonucleotides can be used in pharmaceutical compositions which cause an inhibition of the translation of the androgen receptor and/or the 5α-reductase efficient against acne, possibly in combination with other anti-acne means, in combination and/or mixture with pharmaceutically acceptable drug carriers usual for the respective above-described types of application.

There are suited, for instance, tablets or capsules having the active component(s) together with extending fillers such as, e.g., lactose, dextrose, sucrose, manitol, sorbitol, cellulose and/or glycine, lubricants such as, e.g., silicon dioxide, tallow, stearic acid as well as the magnesium or calcium salts thereof and/or polyethylene glycol, for tablets furthermore binders such as, e.g., magnesium aluminum silicate, starch, gelatin, tragacanth, methyl cellulose, sodium carboxymethyl cellulose and/or polyvinyl pyrrolidon, and, if desired, disintegrating agents such as, e.g., starch or modified starch, agar, algeinic acid and/or the sodium salt thereof, or mixtures thereof, and, where appropriate, absorbents, dyes, flavor additives and sweeteners. Compositions adapted to be injected are, for instance, aqueous isotonic solutions or suspensions, and suppositories are preferably prepared of fat emulsions or suspensions. Local and topical, resp., pharmaceutical compositions are, for instance, ointments, creams, gels, oils, emulsions, lotions, pastes, liposomes or solutions and they contain, apart from the active agents, the additives appropriate to the above-mentioned formulations. In the case of locally topical applications furthermore agents for increasing the percutaneous resorption can be added, for instance hyaluronidates, dimethyl sulphoxide (DMSO) and the like. Said pharmaceutical compositions can be sterilized and/or contain further additives such as preservers, stabilizers, wetting agents, emulsifiers etc. The compositions can be prepared by conventional methods of mixing, granulating or coating. The active agent(s) can be contained in different compositions and quantities from 0.001 to 50% by weight, preferably from 0.01 to 40% by weight, further preferred up to 20% by weight and especially up to 10% by weight referred to the total weight of the pharmaceutical compositions.

Hereinafter the present invention will be explained in detail by way of the following examples.

### EXAMPLE 1

### Treatment of Sebocytes and Keratinocytes With Transfection Reagents in Vitro

Immortalized human sebocytes (SZ95 sebocytes) were kept in a modified DMEM/Ham's F-12 (1:1) medium comprising 10% (v/v) of fetal bovine serum (FCS) and 5 ng/ml recombinant human epidermal growth factor (Biochrom, Berlin, Germany) with 5% of CO₂ and at 37° C. and were employed in passages 50-55.

Primary human epidermal keratinocytes of neonatal prepuce tissue were cultivated in a serum-free keratinocyte medium (Gibco BRL, Berlin, Germany), supplemented by 5 ng/ml recombinant human epidermal growth factor and 50 μg bovine pituitary extract. The keratinocytes were transfected between passages 2-5.

Both the transfection reagents and the oligonucleotides can influence the vitality of the sebocytes and/or keratinocytes. Therefore in the first step the cell cultures were incubated with different transfection reagents in the absence of oligonucleotides so as to test the cytotoxicity thereof:
1. Tfx-10
2. Tfx-20 (Promega)
3. Tfx-30
   The Tfx reagents are a mixture of a synthetic cationic lipid molecule [N,N,N′,N′-tetra methyl-N, N′-bis(2-hydroxyethyl)-2,3-di(oleoyloxy)-1,4-butanediammoniumiodide] and L-dioleoyl phosphatidyl ethanolamine (DOPE). All Tfx reagents contain the same concentration of the cationic lipid component but with different molar ratios of the fusogenic lipid DOPE.
4. DAC-30 (Eurogentec)
   DAC-30 is the liposome form of a mono-cationic cholesterol derivative.
5. DOTAP (Boehringer)
   DOTAP is a liposome form of the cationic lipid N-[1-2,3-dioleoyloxy)propyl]-N,N,N-trimethyl-ammonium-methylsulphate (DOTAP).
6. Effectene (QIAGEN)
7. SuperFect
8. PerFect Lipids (Invitrogen)
   Pfx-1 to Pfx-8 are eight different cationic lipids which differ by their ratio of positive charge per molecular lipid unit.
9. Poly-L-ornithine
10. Polybrene (Sigma)

The transfection experiments are carried out with sebocyte and/or keratinocyte cultures which are confluent no more than 80%, preferably 50 to 70%. The cytotoxic effect of the different transfection reagents was determined with the aid of a LDH assay. This colorimetric test for quantifying cytolysis is based on the measurement of lactate dehydrogenase activity which is released from the cytosol of the damaged cells into the medium supernatant.

The following parameters were varied so as to optimize the transfection efficiency:

a) Serum concentration: The sebocyte medium (modified DMEM/HAM's F-12) is supplemented by different serum concentrations (5%, 2%, serum-free). A serum-free medium was required for an optimum transfection with DOTAP. The toxicity rate doubled in the case of transfection with DAC-30 (4 μg/ml) in the presence of 5% fetal calf serum. DOTAP vesicles showed a moderate cytotoxic effect compared to the other tested transfection reagents such as Tfx-cations. The transfection efficiency checked by fluorescence microscopy was equivalent in a medium containing serum and in a serum-free medium, respectively. The pituitary extract by which the keratinocyte medium is supplemented had no influence on the transfection of the primary keratinocytes with pFx-5 or with poly-L-omithine. However, by reason of a better comparability of sebocytes and keratinocytes also the transfection of keratinocytes was carried out in a pituitary-free medium.

b) Ratio of the Charges of Lipid (L) to the Oligonucleotide (O)

| | Transfection | | | | |
|---|---|---|---|---|---|
| Charge ratio | reagent μm/ml] | | | | |
| L/O | Tfx-10 | -20 | 50 | DAC-30 | DOTAP |
| 2:1 | 26 | 13 | 6 | 2 | 2 |
| 3:1 | 36 | 19 | 8 | 4 | 5 |
| 4:1 | 51 | 27 | 12 | 8 | 10 |

The charge ratio of the transfection reagent turned out to be a critical parameter; the share of the positive charge of the cationic lipid component therefore should be greater than the negative charge share of the antisense oligonucleotides. Random-Primers (ATCG)₅ were used as control oligonucleotides. Low concentrations of DAC-30 (4 μg/ml) and DOTAP (2 μg/ml) in a serum-free medium and in a charge ratio of 2:1 and/or 3:1 showed no cytotoxic effect. Higher quantities of Tfx-10 and Tfx-50 likewise were not lethal, however with the Tfx reagents no sufficient incorporation of the random oligonucleotides in the sebocytes could be detected. The efficiency of the keratinocyte transfection with pFx-5 could be advantageously increased by controlling the oligonucleotide concentration and the lipid quantity. With a decreasing random concentration of 1 μM to 0.5 μM in a pFx-5 ratio of 1:1 the cytotoxic effect could be minimized.

c) Transfection Period (1 h/4 h/24 h/36 h): The transfection periods varied depending on the cell type and the transfection reagent. In the case of the sebocytes the transfection time with DOTAP could be extended from one to 36 hours. The optimum transfection period with DOTAP combined with (ATCG)₅-random primers was 4 hours. Compared to that, the risk of cell damage in the case of DAC-30 was higher and increased significantly in the case of transfection with Tfx-10 after an incubation time longer than 4 hours.

In the case of the primary keratinocytes a transfection could be carried out only with DAC-30, Pfx-5 and poly-L-ornithine, all other transfection reagents turned out to be toxic already after a two hours' incubation. The best results could be achieved by the polycation-L-omithine 12 μg/ml transfection medium and a subsequent DMSO shock (25% DMSO for 4 minutes at room temperature) and by DAC-30 in a charge ratio of 2:1 (3:1). The transfection time could be extended to 24 hours without the cytotoxicity increasing. Polybrene (30 μg/ml) induced a higher rate of cell damage.

Check of the Transfection Efficiency by Fluorescence Microscopy

The cellular uptake of the antisense oligonucleotides is detected by detection of fluorescence in the cytoplasm or the nucleus. The cells were incubated for 24 hours with FITC-(ATCG)₅ under the respective optimum transfection conditions. The sebocytes could be transfected best with DOTAP (charge ratio 2:1). The keratinocytes showed a good FITC incorporation with poly-L-ornithine. In both cell types the transfection efficiency increased proportionally to the transfection period. The cellular fluorescence became stronger and the random oligonucleotides could be detected both inside the nucleus (in the sebocytes) and in the cytoplasm (in the keratinocytes).

Before the transient transfection of the sebocytes and/or keratinocytes with specific antisense oligonucleotides the expression of both target molecules—5α-reductase and androgen receptor—was examined in native SZ95 sebocytes and keratinocytes.

Reverse Transcriptase-polymerase Chain Reaction (RT-PCR)

The RNA detection was made by means of RT-PCR under semi-quantitative conditions (scaling of the mRNA samples with β-actinium). Complete RNA was isolated from cells by a total RNA isolation system (Qiagen, Hilden, Germany). After denaturing (65° C., 5 min) 5 μg RNA were transcribed in cDNA by a ready-to-use T-primed first-strand kit (Amersham Pharmacia, Freiburg, Germany). cDNA fragments of the androgen receptor and of β-actin were amplified with the following primers: β-actin in the reading direction ((5′-AGC CTC GCC TTT GCC GA-3′), reverse direction (5′-CTG GTG CCT GGG GCG-3′); androgen receptor in the reading direction (5′-GAA GAC CTG CCT GAT CTG TG-3′), reverse direction (5′-AAG CCT CTC CTT CCT CCT GT-3′). A total of 2 μl cDNA solution was amplified during 35 cycles by the following program: 1 min at 94° C., 1 min at 60° C. (AR) and 67° C. (β-actin) and 1 min at 72° C. The PCR products were made visible by ethidium bromide dye. β-actin served as an external standard.

Detection of the 5α-reductase and the Androgen Receptor

In the skin primarily the 5α-reductase isoenzyme type-1 is expressed (T_{A} 65° C., Mg 15 mM, product length 369 bp). The androgen receptor expression can be detected using a pair of primers of Exon 2 to Exon 4 (T_{A} 60° C., Mg 25 mM, product length 269 bp).

On the protein level the androgen receptor could be detected by means of intracellular flow cytometry (FACS) in SZ95 sebocytes and keratinocytes. The dyeing with the antibody has to be performed by the intracellular route, because the androgen receptor is localized in the nucleus. The androgen receptor could also be detected by means of Western blot in native SZ95 sebocytes and keratinocytes. After a four hours' transfection of the sebocytes with DOTAP separately and combined with a mismatch-oligonucleotide (5′-CTG CCC TTC AAT CCC TGA GTT TGG C-3′) as well as a subsequent recovery phase of the cells of at least 24 hours, the protein expression of the androgen receptor was comparable to the one provided in the non-transfected cells. With this result a modified (reduced) expression of the androgen receptor can be attributed to a specific antisense oligonucleotide/mRNA interaction.

### EXAMPLE 2

Examination of the Androgen Receptor Expression by Immunoblotting

In the presence of the respective transfection reagent (DOTAP for SZ95 sebocytes and poly-L-ornithine for keratinocytes) SZ95 sebocytes and keratinocytes were transfected for 4 h with different AR-antisense oligonucleotides. The concentration of the examined oligonucleotides was 0.1, 0.4 and 1.0 μM. The inhibiting experiments were carried out in 6-Well plates. After transfection the cells were incubated in their culture medium in order to recover for 14 h, 24 h, 48 h and 72 h. Finally the cells were suspended in the lysis buffer M-PER (Pierce) for the protein extraction. The total protein quantity was determined by the Bradford method. 20 μg of total protein were mixed with a 5-fold test buffer and reduction agent, were heated for 10 minutes to 70° C. and separated in a 3-8% tris-acetate gradient gel. The proteins were transferred upon an Immobilon-PVDF membrane. The blots were blocked overnight with 0.25% casein at 4° C. and then incubated for 1 hour at room temperature, diluted 1/1000 with polyclonal anti-AR antibody from hare (Santa Cruz) for 30 min, and finally diluted 1/10000 with immunoglobulin G directed against hare and conjugated to secondary horse radish peroxidase (Jackson Immuno Research). The blots were evaluated by the chemiluminescence intensification system (enhanced chemiluminescence) by Amersham. The intensity of the AR protein bands was determined densitometrically by the computer program TINA 2.0.

The transient transfection of SZ95 sebocytes with thioate (0.4 and 1.0 μM) and 2′-methylribosyl antisense oligonucleotide (0.4 and 1.0 μM) resulted in a reduced protein expression of the androgen receptor after 24 hours (FIG. 1). After a rather long recovery time the protein expression of the androgen receptor in transfected SZ95 sebocytes again reached the level of the native androgen receptor expression. The most successful transient control of the androgen receptor expression was detected after 14 h with 1.0 μM 2′-methylribosyl antisense oligonucleotide, wherein, compared to native SZ95 sebocytes, an inhibition of 87% of the androgen receptor expression resulted. Thioate antisense oligonucleotide (1.0 μM) resulted in a decrease of the androgen receptor expression of 39% (FIG. 2). These data of the transient transfection of epithelial keratinocytes showed after a recovery period of 17 h a reduction of the androgen receptor expression of approx. 25% compared to native keratinocytes. After 24 h already the androgen receptor level of the treated keratinocytes reached the level of the native keratinocytes again (FIG. 3).

### EXAMPLE 3

The biological activity of the androgen receptor in native and in SZ95 sebocytes transfected with androgen receptor antisense oligonucleotide was examined after stimulation thereof with androgens. SZ95 cells were incubated with testosterone and DHT at concentrations within the range of 1 μM and 0.01 pm. Proliferating effects were measured by the crystal violet assay. By this test, carried out in 24-Well-plates, living cells can be recognized by dyeing with the crystal violet dye. The dyeing intensity correlates with the number of living cells.

Sexual androgens stimulated the SZ95 sebocyte proliferation after 48 h. After 72 h the increase in proliferation was significant at concentrations of 10⁻⁷ and 5×10⁻⁷ M testosterone (FIG. 4) and 10⁻⁷-10⁻⁶ DHT (FIG. 5). Transient transfected SZ95 sebocytes were compared to native cells so as to determine the biological effect of the two different AR-antisense oligonucleotides after androgen stimulation. SZ95 sebocytes were transfected for 4 h with 1 μM of the described phosphorothioate oligonucleotide or 1 μM of the described methylribosyl oligonucleotide. After a recovery period of 14 h the SZ95 sebocytes were incubated with 10⁻⁷ M testosterone or 5-10⁻⁷ M DHT. The control, SZ95 sebocytes merely treated with transfection reagent DOTAP, showed similar proliferation rates as native SZ95 sebocytes (FIG. 6). On the other hand, the thioate-antisense oligonucleotides inhibited the testosterone-induced stimulation of the SZ95 sebocyte proliferation (FIG. 7). The SZ95 sebocytes transfected with the described androgen receptor thioate-antisense oligonucleotide showed the same proliferation pattern as native cells in the presence of testosterone. The inhibition of the testosterone-induced stimulation of the cell proliferation by RNA oligonucleotides was highly significant and probably toxic (apoptosis/necrosis) to SZ95 sebocytes (FIG. 8).

In this way it could be detected on the basis of the above-described examples that the molecularly prepared molecules employed according to the invention (antisense oligonucleotides, ribozymes, stabilized ribozymes, aptamers, mirror aptamers, chimeric RNA/DNA oligonucleotides, naked plasmid DNA, or liposomally encapsulated DNA), especially the antisense nucleotides are highly efficient in bringing about in the human target cells a specific inhibition of the hormonally induced, especially the androgen-induced stimulation of the sebocyte and/or keratinocyte proliferation and therefore are specifically suited and appropriate for the examination and therapy of acne and the acneiform dermaatoses including rosacea.

1. Use of at least one molecular-biologically prepared non-viral active agent selected from antisense oligonucleotides, stabilized ribozymes, aptamers, mirror aptamers, chimeric RNA/DNA oligonucleotides, naked plasmid-DNA or liposomally encapsulated DNA, for the treatment of acne and acneiform dermatoses including rosacea.

2. Use according to claim 1, characterized in that an antisense oligonucleotide having specificity for a nucleic acid target sequence of a gene which codes for steroid hormone receptors or steroid hormone metabolizing enzymes is used.

3. Use according to claim 2, characterized in that the antisense oligonucleotide codes for the androgen receptor and/or the 5α-reductase.

4. Use according to claim 1, characterized in that at least one antisense oligonucleotide is selected from the group consisting of SEQ ID NO:1, SEQ ID NO:2 and degenerated sequences thereof.

5. Use according to claim 1, characterized in that the at least one antisense oligonucleotide is complementary to a ribonucleic acid sequence derived from a gene sequence coding for the androgen receptor and/or the5α-reductase.

6. Use according to claim 5, characterized in that the antisense oligonucleotide is complementary to a single-strand RNA or single-strand DNA or a DNA inside a duplex strand.

7. Use according to any one of claims 1 to 6, characterized in that the antisense oligonucleotide is chemically modified.

8. Use according to claim 1, characterized in that the nucleic acid target sequence is provided in epithelial cells, especially in human epithelial cells.

9. Use according to claim 1, characterized in that the nucleic acid target sequence is provided in cells of the human skin.

10. Use according to claim 8 or 9, characterized in that the nucleic acid target sequence is provided in human sebocytes and/or keratinocytes.

11. Use according to claim 10, characterized in that the target cells are human sebocytes and/or keratinocytes.

12. Use according to any one of the preceding claims, characterized in that the at least one antisense oligonucleotide is introduced into the target cells by liposome-medicated transfection.

13. Use according to any one of the preceding claims, wherein the antisense oligonucleotide is combined with at least one further conventional anti-acne active agent.

14. Use according to any one of the preceding claims, wherein an oral and/or topical application is effected.

15. Use according to any one of claims 11 and 12 for the preparation of a pharmaceutical composition having at least one of the features listed in claims 1 to 10 together with a pharmaceutically acceptable carrier and further adjuvants where appropriate.

16. Use according to any one of the preceding claims for the examination, treatment and/or therapy of acne and acneiform dermatoses including rosacea.

17. Use according to claim 15 for the preparation of products for the examination, treatment and/or therapy of acne and acneiform dermatoses including rosacea.

18. Use of the products obtained according to claim 17 for modifying other cells or for modifying organisms.


SEQUENCE LISTING (7 sequences)

SEQ ID NO: 1 - DNA, 25 bp, Artificial Sequence
  Antisense oligonucleotide
ctgcacttccatccttgagcttggc

SEQ ID NO: 2 - RNA, 25 nt, Artificial Sequence
  Antisense oligonucleotide
gccaagcucaaggauggaagugcag

SEQ ID NO: 3 - DNA, 25 bp, Artificial Sequence
  Mismatch oligonucleotide
ctgcccttcaatccctgagtttggc

SEQ ID NO: 4 - DNA, 17 bp, Artificial Sequence
  Primers for RT-PCR
agcctcgcctttgccga

SEQ ID NO: 5 - DNA, 15 bp, Artificial Sequence
  Primers for RT-PCR
ctggtgcctggggcg

SEQ ID NO: 6 - DNA, 20 bp, Artificial Sequence
  Primers for RT-PCR
gaagacctgcctgatctgtg

SEQ ID NO: 7 - DNA, 20 bp, Artificial Sequence
  Primers for RT-PCR
aagcctctccttcctcctgt